(19) United States
(12) Patent Application Publication
(10) Pub. No.: US 2022/0375582 A1
(43) Pub. Date: Nov. 24, 2022

(54) SYSTEM AND METHOD FOR AUTOMATIC DATA COLLECTION OF PHYSICIAN AND PATIENT INTERACTION TIME

(71) Applicant: ProClock, LLC, Lafayette, IN (US)

(72) Inventors: Clive P. Hohberger, Highland Park, IL (US); Tammy L. Means, Goodland, KS (US); Glenn E. Meiser, Lafayette, IN (US); Norman Means, Goodland, KS (US)

(21) Appl. No.: 17/772,440

(22) PCT Filed: Oct. 24, 2020
(86) PCT No.: PCT/US2020/057243

§ 371 Date: Apr. 27, 2022

Related U.S. Application Data
(60) Provisional application No. 62/926,514, filed on Oct. 27, 2019.

(51) Int. Cl. G16H 40/20; G16H 10/60

(52) U.S. Cl. CPC G16H 40/20; G16H 10/60

(57) ABSTRACT

A system and method for automatic capturing of physician and patient interaction time and location information during provision of medical services by a physician during an appointment with a patient in hospitals, offices or clinics. Capturing total interaction time facilitates verifiable physician reimbursement by third-party payers. RF beacon technology based on the Bluetooth Low Energy devices and communications protocol may be utilized for personnel and location identification. The use of store-and-forward communications enable both reliable interaction-tracking packet data communications to an external host IT system in online offices, and alternatively the queuing of all data for transmission to an off-line host computer.

### CROSS-REFERENCE TO RELATED APPLICATION

This application claims the benefit of U.S. Provisional Patent Application No. 62/926,514 filed on 27 Oct. 2019, hereby incorporated by reference in its entirety.

### BACKGROUND

### Field of the Invention

This invention relates to data collection for interaction tracking. More particularly, the invention relates to the provision of medical services by physicians to patients and the automatic capturing of physician and patient interaction time and location information required for physician reimbursement by third-party payer such as private insurance companies, Medicare, and/or Medicaid.

### Description of the Related Art

Currently, the billing of third-party payers by physicians and other clinicians is a convoluted system based on the complexity of the written medical record, the diagnosis of the patient and the patient contact time spent by the physician.

For example, in the current system, a physician may bill for an appointment time in the 16-30 minutes range. This interaction may have occurred as an initial interaction of 14 mins, and a subsequent interaction of 4 mins (e.g. after laboratory studies were completed), for a total of 18 minutes. This would clearly satisfy billing guidelines for reimbursement as a 16-30 minute visit. However, the insurance auditor might arbitrarily decide that, based on the diagnosis, the visit would only require 10 minutes, and so deny the claim. Currently the physician can offer no irrefutable data proving that the total interaction time with the patient was actually 18 minutes. So, neither side can resolve the insurance claim based on quantitative data, but since Center for Medicare and Medicaid Services (CMS) is more likely to believe and support the findings of its own auditor, the financial loss for 8 additional minutes of time spent may ultimately be absorbed by the physician.

Alternatives exist that allow for billing based on interactive time spent with the patient. However, the difficulty of accurately capturing and documenting such interactive time and the difficulty of proving such time in the face of increasingly punitive government and insurance industry audits, coupled with the resulting “claw back” payments demanded by the auditors when rules appear to have been transgressed, has led to reluctance to use interactive-time based billing. This reluctance is based in large part on the inability to accurately document and record interaction time data in a way that cannot arbitrarily be deemed by auditors to be inaccurate or even fraudulent.

The same desire exists to record interactive time with physician staff, both to measure staff productivity and because certain payments are based on time spent performing procedures by certain skilled-based staff.

This problem has been magnified by the CMS policy of using outside for-profit auditors, so-called “Recovery Audit Contractors (RAC) auditors,” who are paid by commission based on a percentage of collections. One result has been a catastrophic loss of physician productivity, as healthcare entities focus on ever increasing documentation complexity in an attempt to forestall punitive “claw back” payments demanded by RAC auditors, which has led to physicians being dragged into ever-increasing levels of clerical work at the expense of time spent in actual healing. The result is a decline in treatment quality and a further increase in healthcare cost.

To address this explosion of paperwork, the CMS has proposed massive reforms in their billing system. Starting in 2020, CMS has proposed rules which, among other changes, will offer amended options to outpatient visit billing.

The first option is to continue billing based on the current, complex system which bases reimbursement on the amount of documentation entered into the medical record.

As a second option, CMS has proposed an alternative system based on actual “face-to-face” interaction time by the physician with the patient during an outpatient office visit.

Finally, as a third option, CMS has proposed new billing codes which will allow significant payment enhancements based on actual “face-to-face” extended interaction time spent by physicians with patients during the visit, when the total time exceeds a certain minimum amount.

However, CMS has not proposed elimination of the RAC audit system. Since continuing to use the old (i.e., first option) documentation-dependent billing rules will cause healthcare entities to be subject to potentially ruinous and abusive RAC audits, there will be a strong incentive for healthcare providers to switch over to time-based billing.

Without an objective and automatic tracking system which tracks physician-patient interaction time, any switch to time-based billing would still leave healthcare providers subject to possibly unfounded, arbitrary and capricious allegations by RAC auditors of overbilling due to overstatement of actual time spent with the patient, thus potentially resulting in demands from CMS of ruinous “claw back” repayments.

Alternatively, healthcare providers may find that they are actually underbilling for services rendered when using the documentation-based current system; careful and objective tracking of physician-patient interaction times may result in increased billable charges under the new time-based system.

The invention herein comprises an accurate, reliable, and objective system to automatically capture total physician-patient interaction time during a patient appointment, and also staff-patient interaction time. Not only will this make auditing simple and reduce “claw back” payments due to a trackable, fully objective and automatic recording of actual “face-to-face” healthcare provider-patient interactions, this will also result in massive across-the-board direct and indirect savings as physicians and hospitals can reduce clerical time and yet be assured of the stability of payment streams. This invention would allow physicians and hospitals to choose to use and document the time-based billing alternative when such billing would be more remunerative than other alternatives.

### SUMMARY

A data collection system for automatically collecting of physician-patient interaction time data during a patient appointment is disclosed. The system also records physician staff interaction times, and the location of the patient, physician and staff during these interactions. Data is collected as a sequence of events forming a message stream which is processed by an external online host IT system or offline host computer. The host IT system or host computer, neither of which are within the scope of this patent, reconstructs for the physician's billing system the total patient interaction times with physicians and staff members together with treatment location data.

In collecting data for interaction times between a patient, physician (or other provider including the physician staff) in a doctor's office, the one thing that is constant in all interactions is the patient. Therefore, a patient-centered approach is used in automatically collecting interaction time data about the physician and staff with whom the patient interacts, and the locations within the physician's office complex where that interaction occurs.

An important concept here is that this is fundamentally a patient contact and point of care data collection recording system, utilized for the duration of a patient appointment. No real-time decisions are made during the patient appointment based on the recorded data. It is therefore unnecessary and optional to upload any real-time interaction time and location data during the patient appointment for billing purposes.

Electronic Healthcare Record (EHR) and Electronic Medical Care (EMR) systems may also operate to capture patient data in real-time, which this system completely facilitates by capturing the real-time interaction time and location data during the patient appointment. An extension of this system is to upload the captured real-time interaction time and location data in real-time to an EHR or EMR system.

Radio frequency beacon tags used may be Bluetooth Low Energy (BLE) devices, which are representative of beacon tags that each regularly and independently advertise (RF broadcast) a universally unique identifier (UUID) to nearby fixed and portable electronic devices called beacon receivers. This system comprises multiple beacon tag devices each utilizing an RF protocol such as Apple's iBeacon or Google's Eddystone which use BLE hardware to identify people and locations. Tags using the iBeacon protocol only advertise a UUID, while Eddystone offers an extended advertising protocol for limited telemetry.

Alternative Bluetooth devices and/or beacon protocols may be used. Other hardware embodiments including RFID reader systems utilizing active or passive RFID tags in place of beacon tags could be used.

During a visit, the patient wears a Bluetooth LE beacon receiver. The patient's beacon receiver may take many physical forms, including the physical form of a front-facing patient ID badge similar to those ID badges worn by other personnel. ID badges may be, for example, clipped to patient clothing or worn hung from a neck lanyard.

The patient's beacon receiver has the ability to receive and record UUID signals from all the beacon tags in the area around the patient from personnel, location identifiers and special equipment if desired. In addition, the beacon receiver will upload data to the host IT system either in real-time, or off-line after the patient appointment, to enable reconstruction of the total physician and staff contact times and locations during the patient appointment.

When real-time decisions based on the data are not required, the patient's beacon receiver will use store-and-forward messaging to pass sequential messages to an external host IT system. The store-and-forward capabilities allow all the data captured during the appointment to be uploaded to the host IT system after an appointment, as well as facilitating real-time data communication with an online host IT system during an appointment. Thus, this physician-patient interaction time reporting system functions equally well in both online and offline physician office environments.

Since under current and future billing regulations it is possible to bill for services on an actual face-to-face interaction time basis, the use of this system to capture actual physician-patient interaction time will allow for automatic determination by EHR/EMR software systems of the appropriate billing code.

As stated earlier, one strong disincentive for utilizing actual face-to-face interaction time basis billing method has been the lack of objective data that could withstand an audit by third-party payers. By objectively capturing interaction time on a minute-by-minute basis, the interaction time results captured by this invention will be difficult for an auditor to challenge.

Coupled with the upcoming simplification of documentation requirements, the use of this invention would require substantially less time spent with documentation by the physician and/or their staff, thus increasing physician and staff productivity.

### TECHNICAL BACKGROUND

### Beacon Tags

Bluetooth Low Energy (BLE) is a wireless personal area network technology standard designed and marketed by the Bluetooth Special Interest Group (Bluetooth SIG). BLE beacon technology is widely available and aimed at novel applications in the healthcare, fitness, location tracking, security, and home entertainment industries.

Beacon tags as used here are, for example, a class of BLE devices that each independently “advertise” (RF broadcast) a universally unique identifier (UUID). Beacon tags are typically used for identification and locating purposes. Various vendors make BLE beacon tags. As examples, two popular beacon tags are Apple's iBeacon or Googles Eddystone, both of which use BLE operating at 2.45 GHz, but with different message protocols.

Apple's iBeacon licensed protocol was introduced in 2013. iBeacon is based on BLE proximity sensing. The UUID identifier and several bytes sent with it can be used to determine the iBeacon's physical location, track personnel present, or trigger a location-based action.

Google's Eddystone beacon protocol was released by Google in July 2015, and appropriately named after the famous Eddystone Lighthouse in the English Channel. Similar in protocol to the Apple iBeacon, Eddystone also contains a telemetry message (Eddystone-TLM) designed for reporting the beacon tag's health, including the battery voltage and tag temperature.

Most beacon tags have programmable firmware used with their beacon protocols that control several characteristics:

(1) Advertising Interval: The (usually constant) time interval between beacon tag emissions of its UUID signal is its advertising interval. The Apple iBeacon protocol specifies a default advertising interval of 100 ms. Most beacon tag users opt for a longer advertising interval to extend the tag battery life. Since in a physician's office, things happen at human response speeds, here assume a typical default advertising interval of 1000 ms (1 second).

(2) Transmit RF Power: Beacon devices transmit a signal with a preprogramed or otherwise fixed transmission RF power, with BLE beacon tags typically transmitting 0.01 mW to 10 mW. Higher RF power means the signal can travel longer distances. Lower RF power means less battery consumption but also short read range. Balance in the setting the RF power is not just important to ensure long battery life. It is also important in this invention to ensure the patient's beacon receiver can reliably read the beacon tags in the patients' examination room, and also that those same beacon tags are not being detected by other patients in adjacent examination rooms (crosstalk). RF power on personnel tags and room IDs is therefore typically set low to minimize range and prevent crosstalk between locations.

(3) Advertising message: Usually a fixed-length multi-byte structured data string. Content and UUID structure vary with the beacon protocol in use. Here the UUID and additional bytes have been pre-associated to indicate, for example, the beacon's physical location, personnel identification; medical equipment present, and also in certain protocols the beacon battery status.

Each beacon tag advertises a unique error-corrected UUID message with a constant but not necessarily identical advertising interval, nominally in the range of 1 second. As with the general class of “Tag-talks-first” communications protocols, beacon tag intervals are deliberately not synchronized and have randomized advertising intervals to minimize data collisions, so that eventually each tag will be heard clearly. The power of each of the beacon tags is carefully controlled to limit the area around it in which the beacon signal can be detected.

With low transmit RF power and a long advertising interval, the beacon tags are typically powered with a simple 3v Lithium “coin” primary batteries, and have a typical battery life of 1-3 years.

### Physician and Staff Identification

In this physician-patient interaction time reporting system, all physicians and their staff members will each wear a beacon tag with a UUDI uniquely identifying that person. Beacon tags with unique IDs are affixed to identify every location where a patient would meet with a physician or staff member (office, examining room, etc.); receive in-office diagnostic work (such as EKG, X-ray, blood work, etc.); or receive therapy (such as bandage, splint or cast fitting; physical therapy or IV therapy, etc.). In addition, each chaperone optionally in attendance with the patient (such as parents, spouse, etc.) may also wear a visitor's beacon tag uniquely identifying them.

### Beacon Receiver Usage

The patient wears a beacon receiver reading UUIDs from advertising messages from all local beacon tags using the same beacon tag protocol, and recording event packets according to an Event Recording Protocol described herein.

The patient's beacon receiver may be packaged physically like the beacon tag ID cards worn by other personnel, except the beacon receiver has the ability to receive and record UUIDs from all the beacon tags in the area around the patient, and communicate this data forward to an IT system using BLE or the physician's office or hospital Wi-Fi or offline after the patient appointment.

When not yet assigned to a patient use, the beacon receiver may be configured to sit in a special cradle in the patient reception area of the physician's office. The cradle provides several features comprising: optional contact or contactless wireless battery charging; a cradle identification beacon tag; BLE or Wi-Fi data communications port to the online or offline host IT system to upload the collected beacon tag data. It may optionally comprise an automatic locking latch for the beacon receiver when placed in the cradle with an electronic unlock controlled by the beacon receiver cradle through communications with the host IT system.

Once the beacon receiver is inserted in the cradle, it may be RF-isolated from external RF beacon tags. Additionally or alternatively, an internal cradle identification beacon tag may be operative as a flag so that the beacon receiver knows to stop recording while placed in the cradle, and to complete any stored packet data transmission. Detection of the beacon receiver in the cradle may also be recorded as the last event packet in the packet sequence of each appointment message stream. After the last packet of the patient appointment message stream has been successfully sent to the host IT system or offline computer, the packet queue is now empty and the beacon receiver now resets itself for use in a new patient appointment.

When the patient receptionist wishes to assign a beacon receiver to a patient for a new physician appointment, the connected host IT system sends a new patient assignment message to the beacon receiver through the cradle communications. If the beacon receiver has been charged and reset, it confirms to the host IT system acceptance of the new patient assignment, and records the new patient assignment as the first event packet of a new appointment packet sequence. If an optional electronic cradle latch is in use, the host IT system unlatches the beacon receiver for use. Once released, it is assumed assigned to that specific patient and starts recording for the duration of the patient appointment, until re-latched into the cradle at patient checkout—signaling end of the patient appointment.

### Beacon Receiver Event Recording Protocol

During the patient appointment, during each consecutive recording interval (nominally 1 minute) the beacon receiver enters on the corresponding interval beacon list the UUID message of each beacon heard clearly at least once during each recoding interval.

The new list is compared with the list from the prior recording interval. If no beacon tag has been added or subtracted, nor any beacon tag's UUID message changed, then no action need be taken; only changes are reported Eliminating transmission of redundant packets greatly reduces the amount of data which needs to be uploaded to the host IT system.

If the beacon tag list did change, the beacon receiver forms an event packet. The event packet comprises a header and packet data. The header includes the beacon's UUID which has been associated with the patient for the duration of the appointment, and a packet timestamp for the ending time of the recording interval. The beacon receiver also increments the next packet sequence number and includes it in the packet header. The combination of the packet timestamp and packet sequence number in each packet header ensures that all changes to the beacon tags sensed by the patient are recorded reliably in sequential order and transmitted to the host IT system with a minimum number of packet communications (comprising initial packet transmission, acknowledgment by the host, and retransmissions when required). The packet data comprises the UUIDs of all beacons on an interval beacon list, reduced to a single entry for any beacon tags which were received multiple times during the recording interval which have identical UUIDs. The use of timestamp and packet sequence number also ensures data security and traceability, essential attributes necessary in the case of audit.

Utilizing the store-and-forward communications protocol described herein, the beacon receiver then queues each event packet for communication over BLE in the packet queue sequence number order. It also enters the packet sequence number on an unreceived packet number list to indicate that packet receipt has not yet been confirmed by the host IT system.

When online BLE network communications are available, beacon packets are transmitted via BLE to a host IT system for processing. When no real-time communications are available, the packets just remain stored queued in packet sequence order within the beacon receiver memory. This allows all the data captured during the appointment to be to be uploaded to and processed by an offline host computer after the patient appointment is concluded.

### Store-and-Forward Communications Protocol

An issue in an environment with mobile patients is the reliability of short-range on-line communications as the patient moves from room to room within the physician's office or office complex. The beacon receiver already utilizes BLE communication with the beacon tags. However, the fact that Wi-Fi may already be in place to support other wireless connections with the physician's office network means that the use of Wi-Fi rather than BLE for communication between the beacon receiver and the host IT system is a perfectly acceptable alternative.

Use of the store-and-forward communication strategy allows for the possibility that there may be interruptions in the online BLE connection for many reasons, such as the patient moving into an area where the signal is poor, challenging real-time data communication with an online host IT system during an appointment.

To implement store-and-forward communication in the beacon receiver, a communications protocol similar to that followed in satellite communications is adopted. Satellite communications between the Earth station and satellite transceivers are based on a message divided into a sequence of fixed-size or self-defining variable-size data packets. These are transmitted as a message stream in nominal packet sequence. It is assumed that the communications channel is noisy, so that some of individual packets in a packet sequence may not be read correctly at the receiver (either the satellite or the Earth station).

Each packet should utilize the well-known Reed-Solomon error correction capability or the like, to enable the recovery of data at the receiving device from most partially-corrupted packets. Each packet received perfectly, or reconstructed properly through the use of error correction, causes the host computer to send back a confirming RECEIVED acknowledgement with the packet number. That original packet is then deleted from the beacon receiver packet queue and its packet number deleted from the unreceived packet number list.

If the packet data is unable to be recovered through error correction, or when a packet was missed from the received packet sequence, then the communications receiving device asks the transmitting device to RESEND a particular packet identified by that packet's sequence number if available, or else range of packet numbers including the missing packet numbers. Only packet numbers in that range which have not yet been successfully received will be retransmitted.

In the case of Earth-satellite communications, receipt of the RESEND request is at least 260 milliseconds after the packet was originally sent due to the communications path length, so the retransmitted packet may be received out of sequence by hundreds to thousands of intervening good packets. The retransmitted packets, when received correctly, may be reordered into sequence even though received out of sequence through use of the packet sequence. This enables the entire transmitted message stream to be reconstructed in sequential packet order.

If the packet is not received correctly after some defined number N of retransmissions, then the communications receiving device sends the transmitting device a CORRUPTED response message with either the packet number or the ostensible data string of what should have been the packet header. This prevents the store-and-forward communications from looping forever while trying to send a corrupted packet.

All properly-formed packets ideally should eventually be received correctly. The IT host computer then reconstructs and processes the patent appointment message stream contained within the properly-ordered packet sequence, taking appropriate action to deal with data missing from any rare corrupted packets.

When no on-line data communications is available, store-and-forward capabilities also allow all the data captured during the appointment to be to be uploaded to and processed by an offline host computer after the appointment. Thus, this physician-patient interactive contact time reporting system functions equally well in both on-line and offline physician office environments.

### BRIEF DESCRIPTION OF THE FIGURES

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 is a representation of a physician examining a patient in an examination room, according to an exemplary embodiment.

FIG. 2 shows a block diagram of an exemplary beacon receiver.

FIG. 3 shows a block diagram of a beacon receiver placed in an exemplary charging and communications cradle.

### DETAILED DESCRIPTION

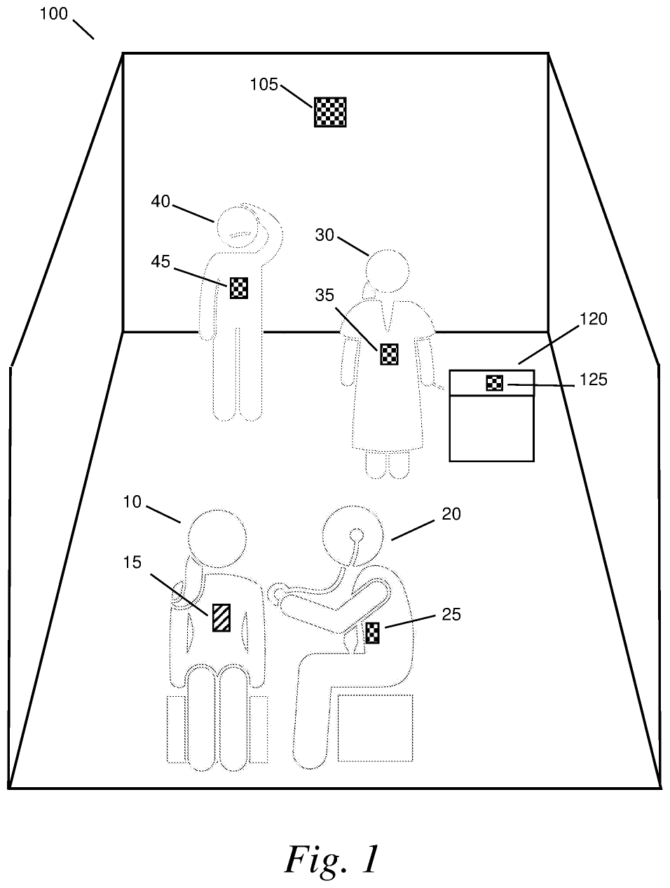

In FIG. 1, a patient 10 is examined by a physician 20 in examination room 100 (only 3 walls of the examination room are shown). If necessary, one or more staff members 30 is in attendance. Optionally, one or more chaperones 40 such as parents or a spouse may also be in attendance with the patient 10.

The physician 20 may be, for example, a Doctor of Medicine (MD), Doctor of Osteopathic Medicine (DO), Physician's Assistant (PA) or Nurse Practitioner (NP), Emergency Medical Technician (EMT) or other licensed practitioner whose services can be submitted for reimbursement to a third-party payer such as an insurance company. The physician 20 may be a primary care Physician and the patient 10 may be a patient of the primary care Physician.

Physician 20 is wearing a unique beacon tag 25, and also each attending staff member 30 is wearing a unique beacon tag 35. Any optional patient chaperone 40 attending is wearing a unique visitor beacon tag 45. Each beacon tag advertises a unique ID number (UUID), associated with the identification of the person wearing it or the object or location identified.

The examination room 100 also has beacon tag 105 advertising a unique ID associated with its treatment location which may be associated with its diagnostic or therapeutic function. Note that any room may have multiple beacon tags depending on its size, each with unique UUIDs. There may be also beacon receiver protocols for the reading of multiple location beacon tags in order to triangulate patient position within the room or building.

One skilled in the art will appreciate that the examination room 100 described herein is a generic representation of a contact location such as a physician's office, a physician's home, a patient's office, a patient's home, a hospice, a nursing home, a conference room, an operating room, or either a patient examining room or a patient treatment room in a clinic, an accident site, or any other location where it is beneficial to record interaction time between a caregiver and a patient.

Additionally, any medical device 120 (e.g. an EKG instrument) present optionally may have a beacon tag 125 advertising a unique ID associated with this device which is associated with the diagnostic or therapeutic function of medical device 120.

Patient 10, however, wears beacon receiver 15 temporarily assigned by the physician's receptionist for the duration of the patient appointment. Beacon receiver 15 receives and records the advertised UUIDs from all beacon tags in proximity to the patient 10. In FIG. 1, patient examining room 100 example comprises personnel beacon tags 25, 35 and 45; location beacon tag 105; and medical instrument beacon tag 125. Note that the beacon receiver 15 may also receive and report stray beacon tag signals from other unassociated nearby personnel and locations.

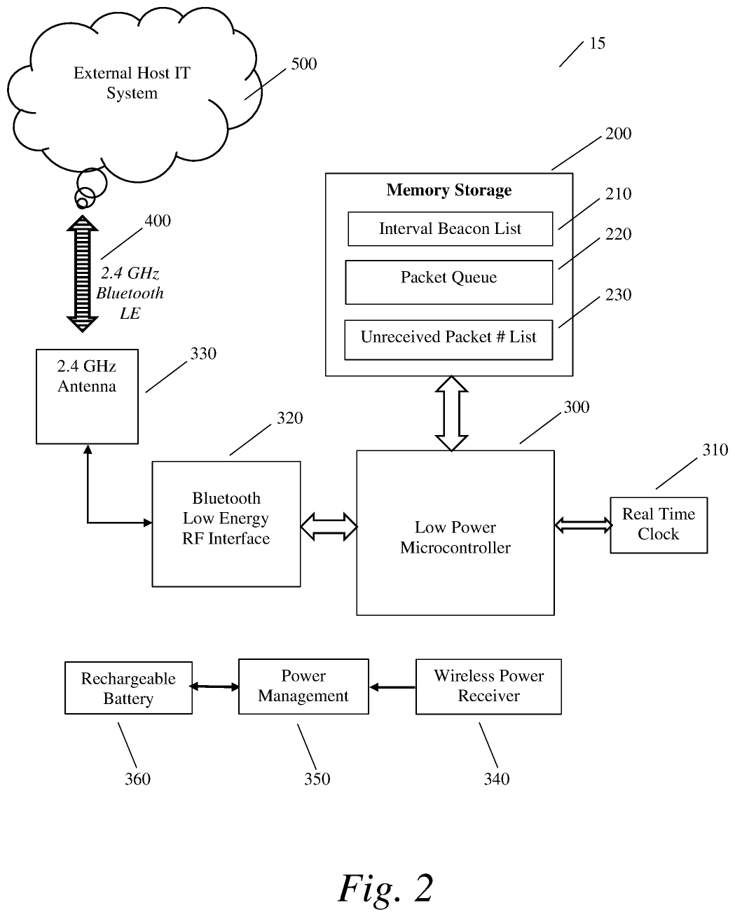

FIG. 2 shows a block diagram of the battery-powered beacon receiver 15. The beacon receiver 15 comprises a memory 200; a low power microcontroller 300 with real-time clock 310 either as an internal or externally attached module; a Bluetooth Low Energy RF interface module 320; and a 2.4 GHz antenna 330. Through antenna 330 the entire beacon receiver 15 is in communications, for example via 2.4 GHz BLE communications 400, with an external host IT system 500.

Note that the beacon receiver 15 may be provided without a wearer accessible on-off switch, to prevent accidental or deliberate nonrecording during part of a patient appointment. The beacon receiver power system in FIG. 2 is comprised of wireless power receiver module 340 connected with power management module 350 and rechargeable battery 360. Power management module 350 controls both the external charging by 340 of rechargeable battery 360 and the internal supply of DC power to the rest of the beacon receiver 15 electronics comprising beacon receiver modules 200, 300, 310, 320 and 330.

At regular recording intervals the beacon receiver 15 records the UUIDs of all tags heard during that interval on an interval beacon list 210 in memory 200. The interval beacon list of beacon UUIDs 210 may change in the current recording interval from that of the prior recording interval. One cause may be personnel entering and leaving the treatment location. Another cause may be the patient moving between locations, such as between a patient examining room and a laboratory for a blood draw. In each time interval that the interval beacon list 210 changes, an event message packet is created with a header comprising a timestamp from real-time clock 310 and an incremented packet sequence number. Each event message packet is queued in packet queue 220 for subsequent transmission to an external host IT system 500. An entry for that packet sequence number is also made on the unreceived packet number list 230.

Each event message packet in packet queue 220 comprises a header and data. The packet data comprises the UUIDs of all beacons on interval beacon list 210 for that recoding interval. Any beacon tags which were received multiple times during the recording interval are consolidated to a single entry. The data may optionally comprise telemetry messages received as part of the beacon receiver data or through additional query and response by the beacon receiver.

The microcontroller 300 manages the store-and-forward communication protocol for transmission of the packets in packet queue 220 in the nominal order identified on unreceived packet number list 230. Once the 1^{st} packet is queued, the microcontroller 300 causes a query to determine if RF bidirectional communications 400 to the host IT system 500 is active. If communications are available, data transmission starts with the lowest number packet on the unreceived packet number list 230.

If successful receipt of the packet sequence number is acknowledged by a RECEIVED message from the host IT system 500, then that packet number is deleted from the unreceived packet number list 230 and the packet itself is deleted from the packet queue 220.

If a RESEND request is received from host IT system 500, either for a single packet sequence number or a range of packet sequence numbers (typically caused by a short dropout or noise burst in communications), then that single packet or any unreceived packets in the requested packet number range are retransmitted to the host IT system 500 until successfully received and acknowledged, or that packet is declared corrupted by the host IT system after a specified number of attempts, N, in which case that packet is deleted from the unreceived packet number list 230 and the packet itself is deleted from the packet queue 220.

The process continues until all uncorrupted packets in the patient appointment message stream have been sent and the unreceived packet list and packet queue are both empty.

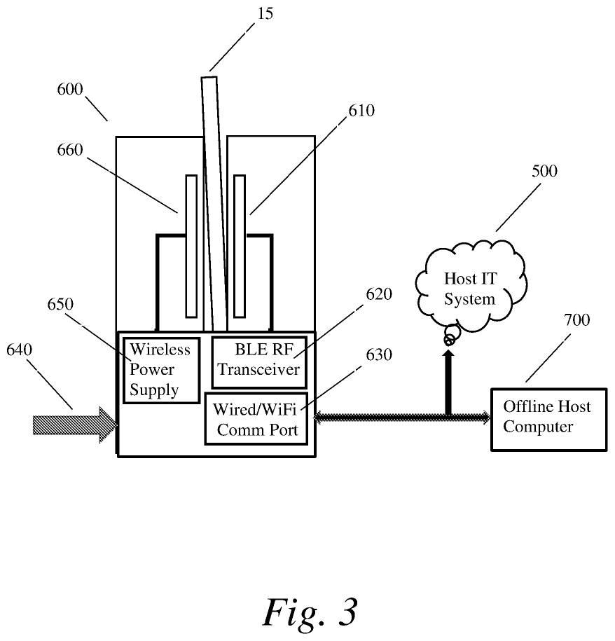

When the patient returns the beacon receiver 15 to the physician's receptionist, the beacon receiver 15 is simply placed in cradle 600 shown in FIG. 3. The cradle 600 comprises a BLE transceiver 620 functionally similar to the system shown in FIG. 2, and a wireless power supply 650 with charging antenna 660 for the rechargeable battery 360 in beacon receiver 15, and external wired or wireless communications port 630 to either optional online host IT system 500 and/or optional offline host computer.

External power 640 is used to support wireless power charging system 650 and BLE transceiver 620. If communications port 630 is an Ethernet connection, then power-over-Ethernet optionally may be utilized in place of external power 640 to support the wireless power charging system 650 which uses wireless power antenna 660. Charging of the beacon receiver 15 rechargeable battery 360 takes place independently of any store-and-forward packet communications of the patient appointment message stream from beacon receiver 15.

If online communications of the patient appointment message stream to an existing host IT system 500 was not complete prior to the beacon receiver 15 being placed in cradle 600, then packet communication continues with the host IT system 500 until completed, but now through BLE antenna 610 connected to the cradle BLE transceiver 620. In this manner store-and-forward communications of all event packets of the patient appointment message stream stored in 220 is assured.

Alternatively, communications and/or charging between the beacon receiver 15 and the cradle 600 may be via electrical contacts of the devices that mate upon insertion of the beacon receiver 15 into the cradle 600.

The data collection systems specified in this invention also works equally well in offline environments where no online communication is available. If no online communication of the patient appointment message data stream was feasible when the beacon receiver 15 is placed in cradle 600, the BLE transceiver 620 now forwards that entire patient appointment message stream queued in 220 directly to an offline host computer 700. This is performed using wired or wireless communications port 630 and utilizing the same store-and-forward packet data transmission protocol as would be used with an online host IT system 500.

Once all event packets of the patient appointment message stream have been transmitted to either online host IT system 500 and/or offline host computer 700 the beacon receiver 15 resets and is now available for assignment to another patient, if a minimum level of rechargeable battery 360 charge is indicated by the beacon receiver 15.

One skilled in the art will recognize that the present inventions enable collection of detailed patient appointment interaction data which facilitates verifiable billing in a range of billing protocols acceptable to various third-party payers, freeing the physician and/or their staff from time consuming administrative procedures that are themselves unbillable time.

The collected data may be further utilized to automate generation of patient records, such as an Electronic Medical Record or an Electronic Healthcare Record.

Where in the foregoing description reference has been made to ratios, integers, components or modules having known equivalents then such equivalents are herein incorporated as if individually set forth.

While the present invention has been illustrated by the description of the embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the invention in its broader aspects is not limited to the specific details, representative apparatus, methods, and illustrative examples shown and described. Accordingly, departures may be made from such details without departure from the spirit or scope of applicant's general inventive concept. Further, it is to be appreciated that improvements and/or modifications may be made thereto without departing from the scope or spirit of the present invention as defined by the following claims.

We claim:

1. A method for recording patient interaction time, comprising the steps of:
associating a beacon receiver with a patient and coupling the beacon receiver to the patient;
associating a plurality of beacon tags with each of a physician, a staff and a location to be used during a patient interaction;
each of the beacon tags configured to broadcast a beacon signal including a unique identifier, according to a selected time interval;
logging beacon signals received by the beacon receiver from the beacon tags associated with the physician, the staff, the location and/or the equipment identifiers during a patient interaction in an interaction log; and
transmitting the interaction log to a host computer.

2. The method of claim 1, wherein the beacon receiver and each of the beacon tags utilize Bluetooth Low Energy communication protocol.

3. The method of claim 1, wherein the transmission of the interaction log is performed at an end of the patient interaction.

4. The method of claim 1, wherein the transmission of the interaction log is performed during the patient interaction.

5. The method of claim 1, wherein the transmission of the interaction log is performed upon return of the beacon receiver to a cradle configured for communication with the beacon receiver and electrical charging of the beacon receiver.

6. The method of claim 5, wherein the cradle communication with the beacon receiver is via Bluetooth Low Energy communications protocol and the electrical charging of the beacon receiver is wireless.

7. The method of claim 1, wherein the transmission of the interaction log is performed utilizing a store-and-forward packet data transmission protocol.

8. A system for automatically recording the total length of continuous or discontinuous interaction time between a physician and a patient during a patient appointment, wherein interaction time is defined as the time period during which both are in continuous visual or speaking proximity in a contact location, and the total interaction time is recorded in either a physician record or a patient record.

9. The system of claim 8 wherein the contact location is also recorded in either a physician meeting record or a patient meeting record.

10. The system of claim 8 wherein the contact location is selected from the group consisting of physician's office, physician's home, patient's office, patient's home, hospice, nursing home, a conference room, an operating room, or either a patient examining room or a patient treatment room in a clinic or hospital, or at an accident site.

11. The system of claim 8 wherein the contact location is within a single room or a predefined area within a larger room.

12. The system of claim 8 wherein the contact location may change as various healthcare services are rendered during the patient appointment.

13. A method for automatically recording the total length of contact time between a physician and a patient during an encounter comprising:
means for automatic identification of both the uniquely identified physician and the patient and any optional physician staff or optional patient chaperone present when both the physician and the patient are in a contact location;
means for measuring and recording the length of each continuous contact time segment when each of the physician and the patient and any optional physician staff or optional patient optional chaperone are present within said contact location; and
means for recording the length of each continuous contact time segment length and contact location and any optional physician staff or optional patient chaperone present in a physician's and/or a patient's record.

14. The method of claim 13 wherein the automatic identification means for identifying the physician comprises a Bluetooth Low Energy (BLE) beacon tag comprising a unique identifier (UUID).

15. The method of claim 13 wherein the automatic identification means for identifying any optional physician staff comprises a Bluetooth Low Energy (BLE) beacon tag comprising a unique identifier (UUID).

16. The method of claim 13 wherein the automatic identification means for identifying any optional patient chaperone comprises a Bluetooth Low Energy (BLE) beacon tag comprising a unique identifier (UUID).

17. The method of claim 14 wherein a physical carrier for the uniquely encoded Bluetooth Low Energy beacon tag is selected from the group consisting of a wearable identification badge, a self-adhesive label or a wristband.

18. The method of claim 15 wherein a physical carrier for the uniquely encoded Bluetooth Low Energy beacon tag is selected from the group consisting of a wearable identification badge, a self-adhesive label or a wristband.

19. The method of claim 16 wherein a physical carrier for the uniquely encoded Bluetooth Low Energy beacon tag is selected from the group consisting of a wearable identification badge, a self-adhesive label or a wristband.

20. The method of claim 13 wherein the automatic identification means for identifying the patient comprises a Bluetooth Low Energy beacon tag reader comprising a unique identifier for the patient.
